(19) United States
(12) Patent Application Publication
(10) Pub. No.: US 2025/0235272 A1
(43) Pub. Date: Jul. 24, 2025

(54) SIMULATED ABLATION TREATMENT PLANS FOR ENERGY DELIVERY SYSTEMS

(71) Applicant: Neuwave Medical, Inc., Madison, WI (US)

(72) Inventors: Scott Laran JOHNSON, Madison, WI (US); James Michael STITT, Madison, WI (US); Sek U. CHEONG, Madison, WI (US); Kevin James WOLETZ, Janesville, WI (US); Barbara Paige HASTINGS, Cape Coral, FL (US); Michael ELSER, New Hope, PA (US)

(73) Assignee: Neuwave Medical, Inc., Madison, WI (US)

(21) Appl. No.: 18/417,479

(22) Filed: Jan. 19, 2024

(51) Int. Cl. A61B 34/00; A61B 18/00; A61B 18/18; A61B 34/10; A61B 90/00

(52) U.S. Cl. CPC A61B 34/25; A61B 18/1815; A61B 2018/00577; A61B 2018/00904; A61B 2018/1869; A61B 2034/107; A61B 2090/364

(57) ABSTRACT

A method of planning a microwave ablation procedure includes uploading a pretreatment scan to a control system of an energy delivery system, displaying the pretreatment scan on a display of the control system, and analyzing the pretreatment scan and identifying a target tissue region that includes a disorder. The method further includes creating and graphically displaying a digital target area on the pretreatment scan, planning and graphically depicting on the pretreatment scan a target path for one or more planned ablation probes, and creating a planned ablation zone for each planned ablation probe based on user-selected parameters. The method further includes placing one or more actual ablation probes in the subject patient, obtaining a probe scan of the subject patient that detects the actual ablation probes, and assigning the planned ablation zone for the planned ablation probes to the actual probes.

### BACKGROUND

Ablation is an important therapeutic strategy for treating certain tissues such as benign and malignant tumors, cardiac arrhythmias, cardiac dysrhythmias and tachycardia. Some ablation systems utilize radio frequency (RF) energy as the ablating energy source. However, RF energy has several limitations, including the rapid dissipation of energy in surface tissues resulting in shallow “burns” and failure to access deeper tumor or arrhythmic tissues. Another limitation of RF ablation systems is the tendency of eschar and clot formation on the energy emitting electrodes, which limits further deposition of electrical energy.

More recently, microwave energy is being used as the ablating energy source in ablation systems. Microwave energy is an effective energy source for heating biological tissues, and is used in applications such as cancer treatment and preheating of blood prior to infusions. One advantage of microwave energy over RF is the deeper penetration into tissue, insensitivity to charring, lack of necessity for grounding, more reliable energy deposition, faster tissue heating, and the capability to produce much larger thermal lesions than RF, which greatly simplifies the actual ablation procedures.

Improved systems and devices for delivering microwave energy to tissue regions is desired. Moreover, since accurate placement of energy delivery devices within a subject patient is critical to successful ablation procedures, systems and devices capable of pre-planning or simulating percutaneous delivery of microwave energy to a subject's tissue is also desired, which can maximize an ablation procedure.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example energy delivery system that may incorporate the principles of the present disclosure

FIG. 2 is a schematic diagram of an example energy delivery device that may be used in accordance with the principles of the present disclosure. according to one or more embodiments.

FIG. 3 is an example display of an example pretreatment scan of a subject patient.

FIG. 4 is an enlarged view of the pretreatment scan of FIG. 3.

FIG. 5 is an enlarged user interface that may form part of the ablation simulation platform, according to one or more embodiments.

FIGS. 6A and 6B are enlarged views of the pretreatment scan of FIG. 4 depicting 2D and 3D planned ablation zones, respectively.

FIG. 7 is an enlarged view of an example second scan, according to one or more embodiments.

FIG. 8 is an updated version of the user interface of FIG. 5.

FIG. 9 is another depiction of the display of FIG. 3 and the pretreatment scan, according to one or more additional embodiments.

### DETAILED DESCRIPTION

The present disclosure is related to systems and methods for delivering energy to tissue for ablation operation and, more particularly, to systems and methods that allow a physician to simulate and plan placement of one or more ablation probes prior to physically placing the ablation probes.

The systems and methods described herein provide a user (e.g. a physician, a surgeon, etc.) with the ability to design and optimize a simulated ablation treatment plan by graphically depicting what a planned ablation will look like, based on a proposed orientation/positioning of one or more probes, and estimating the size of planned ablation zones over the target tissue region. Once the simulated ablation treatment plan is created, the physician may then proceed to place physical probes in the subject patient in accordance with the simulated ablation treatment plan and transfer the planned ablation parameters set for the simulated ablation probes to the actual ablation probes.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof. More particularly, the present disclosure describes systems and devices for pre-planning or simulating the delivery of energy (e.g., microwave energy) to a tissue region (e.g., a tumor, an organ, a blood vessel, foot, lung, bone, etc.).

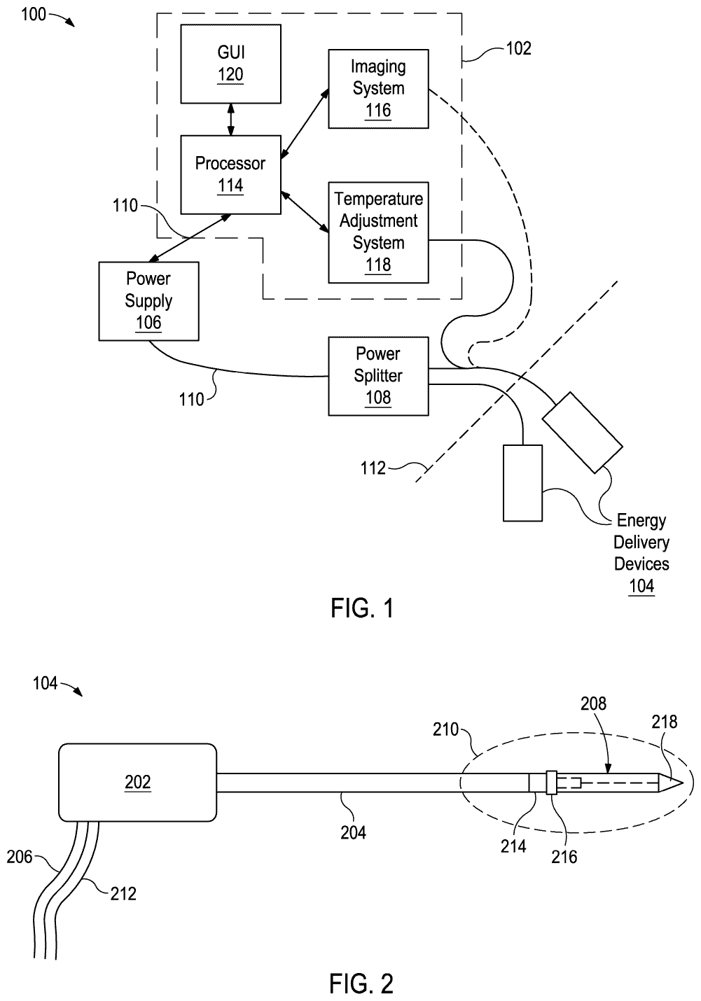

FIG. 1 is a block diagram of an example energy delivery system 100 that may incorporate the principles of the present disclosure. As illustrated, the energy delivery system 100 (hereafter “the system 100”) includes a control system 102 and one or more energy delivery devices or “ablation probes” 104 (two shown) designed to deliver (emit) energy to a target tissue region of a patient. The system 100 further includes a power supply 106 communicably coupled to the control system 102 and the ablation probes 104 to provide electrical power thereto. In some applications, the system 100 may further include a power splitter 108 interposing the ablation probes 104 and the power supply 106 and configured to direct, control, and deliver power to the ablation probes 104.

The components of the system 100 are connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

The control system 102 is configured to monitor, control, and provide feedback concerning operation of the system 100. As illustrated, the control system 102 includes at least a processor 114, an imaging system 116, and a temperature adjustment system 118. The control system 102 may further include a graphical user interface (GUI) 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region for operating and/or monitoring the components of the system 100.

The processor 114 may be provided within a computer system or module, which may include software or software instructions executable by the processor 114 to carry out functions and operations of the system 100. The software may be stored on a computer memory or computer memory device comprising any storage media readable by the processor 114. Examples of the computer memory include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 114. Examples of computer readable media include, but are not limited to, optical discs, magnetic disks, magnetic tape, solid-state media, and servers for streaming media over networks.

Based on instructions provided by the software, the processor 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The processor 114 interacts with the ablation probes 104 to raise or lower (e.g., tune) the amount of energy delivered to the tissue region. The processor 114 may also be configured to prime coolants for distribution into the ablation probes 104 such that the coolant is delivered at a desired temperature. In some applications, the type of tissue being treated is inputted into the software for purposes of allowing the processor 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected ablation sizes (as described in more detail herein) may be based on tissue type. In such embodiments, the processor 114 may not control power delivery based on tissue type. In yet other embodiments, the processor 114 generates a chart or diagram based upon a particular type of tissue or tissue region displaying characteristics useful to a user of the system. The processor 114 may allow a user to choose power, duration of treatment, different treatment algorithms for different tissue types, simultaneous application of power to multiple probes 104, coherent and incoherent phasing, etc. The processor 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

According to embodiments of the present disclosure, and as described in more detail herein, the control system 102 may include a software package including instructions that are executable by the processor 114 to allow a user to input parameters of the tissue to be treated (e.g., type of tumor or tissue section to be ablated, size, location, location of vessels or vulnerable structures, and blood flow information). The software may also allow the user to generate a desired or pre-planned ablation zone on a computed tomography (CT) or other image displayed on the GUI 120 to provide the desired results. Based on the pre-planned ablation zone, the ablation probes 104 may subsequently be positioned at the tissue region and the processor 114 facilitates the ablation zone based on the information provided. Such applications may also incorporate real-time feedback. For example, CT, magnetic resonance imaging (MRI), positron emissions tomography (PET), or ultrasound imaging or thermometry may be used during the ablation process, and this data is fed to the control system 102, which may be configured to adjust various parameters to produce the desired result.

FIG. 2 is a schematic diagram of an example probe 104 that may be used in accordance with the principles of the present disclosure. according to one or more embodiments. As indicated above, the ablation probe 104 may be configured to deliver (emit) energy (e.g., microwave energy, radiofrequency energy, radiation energy) to a target tissue region. As illustrated, the ablation probe 104 includes a handle 202 and an elongate shaft or probe cannula 204 extending distally from the handle 202.

A cable or cable assembly 206 may be operatively coupled and configured to convey electrical power to the handle 202. The cable assembly 206 may extend from the power supply 106 (FIG. 1), for example, and may provide the power sufficient to operate the ablation probe 104. An antenna 208 is provided at the distal end of the probe cannula 204 and receives electrical power from the cable assembly 206 to emit energy to a target tissue region and thereby generate an ablation zone 210. The material of the antenna 208 is durable and provides a high dielectric constant. In some applications, the material of the antenna 208 is zirconium and/or a functional equivalent of zirconium. In at least one application, the ablation probe 104 includes two or more separate antennae 208 attached to the same or different power supplies.

In some applications, a cooling tube 212 is operatively coupled and configured to convey a cooling fluid to the handle 202. The handle 202 may be configured to control conveyance of the cooling fluid into and out of the probe cannula 204 to help regulate a temperature of the antenna 208 and the ablation zone 210. Example cooling fluids include, but are not limited to, water, glycol, air, inert gasses, carbon dioxide, nitrogen, helium, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

In some applications, the ablation probe 104 may include a stick region 214, alternately referred to as a “tissue-loc” region, provided on the probe cannula at or near the antenna 208. The stick region 214 is designed to attain and maintain a temperature that accommodates adherence of a tissue region onto its surface. More specifically, the stick region 214 may operate as an anchoring element having a circulating agent or “coolant” (e.g., a gas delivered at or near its critical point; CO₂) that freezes the interface between the stick region 214 and the adjacent tissue, thereby sticking (maintaining, locking, etc.) the antenna 208 in place during operation. The coolant may be provided to the stick region 214 via the cooling tube 212, for example. Once a pre-determined low temperature is reached at the stick region 214, contact with adjacent tissue causes the tissue to adhere to the stick region 214, thereby resulting in attachment of the energy delivery device 204 to the tissue. During ablation, as the tissue warms, the antenna 208 remains secured to the tissue region due to tissue desiccation and charring. The stick region 214 may be made of any material able to attain and maintain a temperature such that upon contact with tissue induces adherence of the tissue onto the stick region 214. Example materials for the stick region 214 include, but are not limited to, a metal, a, or any combination thereof.

In some applications, the ablation probe 104 may further include a plug region 216 provided on the probe cannula 204 at or near the antenna 208. In at least one application, as depicted, the plug region 216 may be provided distal to the stick region 214 and otherwise interposing the stick region 214 and the antenna 208. The plug region 216 may be configured to prevent a reduction in temperature resulting from the cooled probe cannula 204 and the stick region 214 from affecting (e.g., reducing) the temperature within the antenna 208. Accordingly, the plug region 216 separates interior portions of the ablation probe 104 to prevent cooling or heating of a portion or portions of the device 104 while permitting cooling or heating of other portions. The plug region 216 may be made of an insulative material capable of being in contact with a material or region having a low temperature without having its temperature significantly reduced. Example insulative materials for the plug region 216 include, but are not limited to, a synthetic polymer (e.g., polystyrene, polyicynene, polyurethane, polyisocyanurate), aerogel, fibre-glass, cork, or any combination thereof.

In some applications, the ablation probe 104 may further include a sharp stylet tip or “stylet” 218 positioned at the distal end of the antenna 208 and otherwise forming the distal end of the ablation probe 104. The stylet 218 is designed to facilitate percutaneous insertion of the ablation probe 104. The stylet 218 may be made of a variety of rigid or hardened materials including, but not limited to, a hardened resin, a metal (e.g., titanium or an equivalent of titanium, stainless steel, etc.). In at least one application, the stylet 218 may be braised to zirconia or an equivalent of zirconia. In such applications, the stylet 218 may comprise an extension of a metal portion of the antenna 208 and may be electrically active.

In some applications, the ablation probe 104 may have a coaxial transmission line positioned within the antenna 208, and a coaxial transmission line connecting with the antenna 208. In other embodiments, the ablation probe 104 may comprise a triaxial microwave probe with optimized tuning capabilities. The ablation probe 104 may be the same as or similar to any of the energy delivery devices described in U.S. Pat. No. 11,638,607, issued on May 2, 2023, the contents of which are hereby incorporated by reference in their entirety.

Referring again to FIG. 1, the control system 102 further includes the imaging system 116, which is in communication with the processor 114 comprises one or more imaging devices. Example imaging devices include, but are not limited to, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

In some applications, the system 100 provides software configured for use with imaging equipment of the imaging system 116, such as CT, MRI, and ultrasound, and generate two-dimensional (2D) and or three-dimensional (3D) images viewable by a user on the GUI 120. In some embodiments, the imaging equipment software allows a user to make predictions based upon known thermodynamic and electrical properties of tissue, vasculature, and location of the antenna(s) 208 (FIG. 2). In some embodiments, the imaging software allows the generation of a 2D or 3D map of the location of a tissue region (e.g., tumor, arrhythmia), location of the antenna(s) 208, and to generate a predicted map of the ablation zone 210 (FIG. 2).

In some applications, the imaging system 116 may be configured to monitor ablation procedures, such as monitoring the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104. Likewise, and as described in more detail herein, an algorithm may be applied to predict the shape and size of the tissue region to be ablated (e.g., tumor shape) such that the system recommends the type, number, and location of ablation probes to effectively treat the region.

The temperature adjustment system 118 may be configured to use coolant systems and cooling fluids to help reduce undesired heating within and along the ablation probes 104. In particular, the temperature adjustment system 118 may include the cooling tube 212 (FIG. 2) and may communicate with the handle 202 (FIG. 2) of each probe 104 to control conveyance of the cooling fluid into and out of the probe cannula 204 (FIG. 2), and thereby help regulate a temperature of the antenna 208 (FIG. 2) and the ablation zone 210 (FIG. 2). In some applications, the temperature adjustment system 118 may also be configured to communicate with the handle 202 to operate the stick region 214 and thereby attain and maintain a temperature that accommodates adherence of tissue onto its surface.

The temperature adjustment system 118 may also be configured to continuously or intermittently monitor the real-time temperature of the ablation probes 104. In such embodiments, the temperature adjustment system 118 may communicate with one or more temperature sensors (e.g., thermocouples) terminating at various points along the probe cannula 204 (FIG. 2) and/or the antenna 208 (FIG. 2) of the ablation probe 104. Consequently, localized temperature may be monitored at several points along the antenna 208 to estimate ablation status, cooling status, or safety checks. In some applications, monitoring the temperature at several points along the antenna 208 may help determine the geographical characteristics of the ablation zone 210 (FIG. 2), such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length.

The temperature adjustment system 118 may also be configured to monitor the temperature of a tissue region (e.g., tissue being treated, surrounding tissue). This may prove advantageous in helping to determine the status of the procedure (e.g., the end of the procedure). The temperature adjustment system 118 may communicate with the processor 114 to provide real-time temperature information to a user and display such measurements on the GUI 120. In at least one embodiment, based on the temperature data obtained by the temperature adjustment system 118, the processor 114 may be configured to autonomously adjust operation of the system 100 appropriately.

The power supply 106 may be configured to supply the energy required to operate the system 100. The power supply 106 is also configured to supply energy to the ablation probes 104, such as microwave energy, radiofrequency energy, radiation, cryo energy, electroporation, high intensity focused ultrasound, or any combination thereof. In accordance with principles of the present disclosure, the power supply 106 supplies microwave energy to the ablation probes 104 for purposes of tissue ablation. More specifically, power may be supplied to the ablation probes 102, but the microwave energy is generated in a microwave generator and sent to the antenna 208. In some applications, the power supply 106 may include one or more energy generators configured to provide as much as 100 watts of microwave power of a frequency of from 915 MHz to 5.8 GHz, although the present invention is not so limited. The power splitter 108 may comprise a power distribution system operable to distribute the energy from the power supply 106 to the ablation probes 104. The power splitter 108 may be configured to provide varying energy levels to different regions of the ablation probes 104.

Briefly, conventional percutaneous ablation procedure entails first obtaining or generating a pretreatment scan of a subject patient. The pretreatment scan may be obtained (generated) from a variety of scanning or imaging technologies including, but not limited to, magnetic resonance imaging (MRI), computed tomography (CT) imaging, positron emission tomography (PET), ultrasound imaging, nuclear medicine imaging, fluoroscopy imaging, or any combination thereof. Upon review of the pretreatment scan, a disorder or abnormality located in a target tissue region may be identified, and a determination may be made by a trained surgeon or physician that the target tissue region should undergo an ablation procedure to potentially remedy the disorder. Example disorders or abnormalities can include a tumor or an arrhythmia associated with tissue, an organ, a blood vessel, bone, a gland, or any combination thereof.

In the past and currently, physicians and surgeons undertake ablation procedures by inserting one or more ablation probes into the subject patient, and advancing the distal end of the ablation probe(s) toward the target tissue region where the disorder is located. The physician places the probe(s) based on their knowledge and experience, and with operational knowledge of the ablation probe(s); i.e., best path to reach the target tissue region, whether there are vital organs or vessels in the projected pathway, the projected growth of the ablation zone, the amount of surface area that the resulting ablation will cover, etc. Once locating the target tissue region, the ablation probe(s) may then be operated to heat and kill the tissue located in the target tissue region.

According to embodiments of the present disclosure, systems and methods are provided that allow a physician to simulate and plan placement of one or more ablation probes prior to physically placing the ablation probes. The systems and methods described herein provide a user (e.g. a physician, a surgeon, etc.) with the ability to design and optimize a projected (simulated) ablation treatment plan by graphically depicting what a planned ablation will look like and estimating the size of planned ablation zones over the target tissue region. Once the ablation treatment plan is created, the physician may then proceed to place physical probes in the subject patient in accordance with the ablation treatment plan and transfer the planned ablation parameters set for the simulated ablation probes to the actual ablation probes. The orientation and spatial position of the physical probe(s) may be automatically detected based on imaging. In plans requiring multiple probes, the spatial relationship between planned and physical probes is computed. Ablation parameters are transferred from a planned probe to the physical probe which most closely matches its position and orientation.

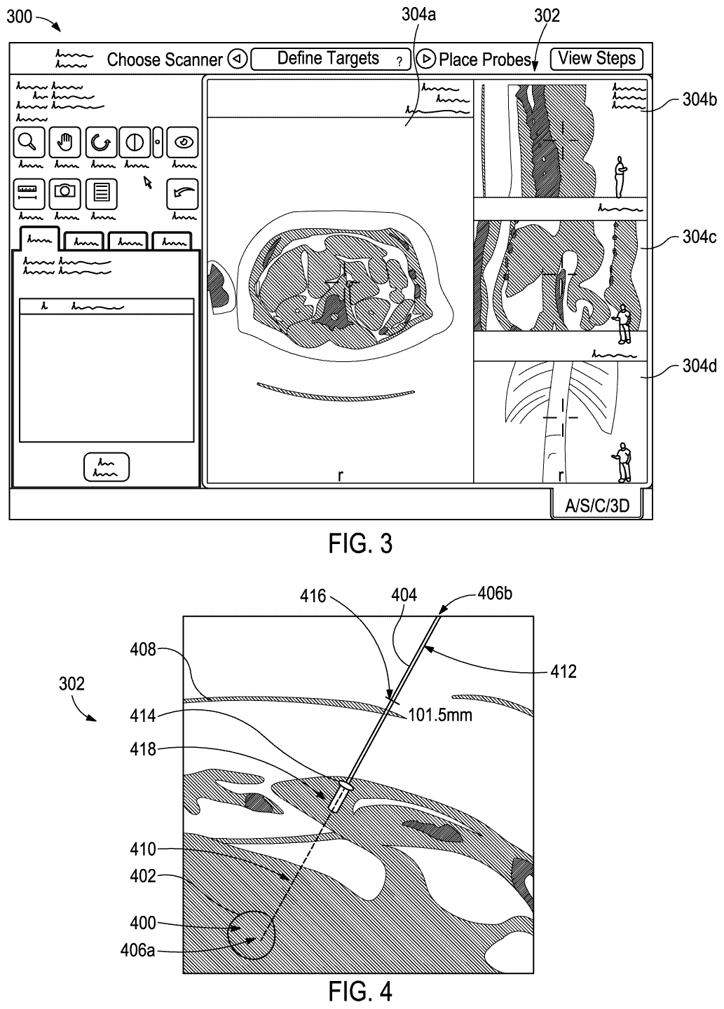

FIG. 3 is a display 300 of an example pretreatment scan 302 of a subject patient. As indicated above, the pretreatment scan 302 may be obtained from a variety of scanning or imaging technologies including, but not limited to, MRI, PET, CT imaging, ultrasound imaging, nuclear medicine imaging, fluoroscopy imaging, or any combination thereof. In some embodiments, the pretreatment scan 302 may be obtained in real time by subjecting the subject patient to the scanning and imaging process. In some applications, multiple pretreatment scans 302 can be obtained and used simultaneously, and in such applications, a user can “lock” or “pair” them together manually to enable the user to scroll through the planes at the same time as they maintain their spatial relationship in the axial plane. In other embodiments, however, the pretreatment scan 302 may be previously obtained and stored in a picture archive system (PACS), which comprises a database or archive system where radiological images are stored in a hospital database. In such embodiments, the pretreatment scan 302 may be accessed and otherwise downloaded from the PACS.

Whether generated in real-time or obtained from PACS, the pretreatment scan 302 may be transferred to an ablation simulation platform programmed and otherwise configured to allow a physician to preplan and simulate an ablation procedure. The ablation simulation platform may comprise a computer product including software instructions stored on a memory or computer readable medium that, when executed (run) by the processor 114 (FIG. 1), causes portions of the control system 102 (FIG. 1) to operate. Accordingly, the display 300 may form part of or be shown on the GUI 120 (FIG. 1).

In some embodiments, as illustrated, the pretreatment scan 302 may include multiple images depicting multiple orientations and depths of the pretreatment scan 302 with respect to the subject patient lying prostrate (horizontal to the ground) during the scanning and imaging procedure. Relative to the scanner, the patient may be oriented supine, prone, or decubitus and feet or headfirst. As illustrated, the pretreatment scan 302 can include at least an axial orientation 304a, a sagittal orientation 304b, a coronal orientation 304c, and a skeletal orientation 304d.

The axial orientation 304a comprises an end view of the subject patient taken along a plane extending vertically through the subject patient and perpendicular to a longitudinal axis extending lengthwise (e.g., from head to toe) through the subject patient. In particular, the axial orientation 304a comprises a vertical layer or “slice” view through the subject patient at a point between the head and the feet. The sagittal orientation 304b comprises a side view of the subject patient taken along a plane extending vertically through the subject patient and parallel to the longitudinal axis. In particular, the sagittal orientation 304b comprises a vertical layer or “slice” view through the subject patient at a point between the left and right sides of the subject patient. The coronal orientation 304c comprises a top view of the subject patient taken along a plane extending horizontally through the subject patient and parallel to the longitudinal axis. In particular, the coronal orientation 304c comprises a horizontal layer or “slice” view through the subject patient at a point between the top (e.g., chest) and the bottom (e.g., back) of the subject patient. The skeletal orientation 304d comprises a graphical depiction of skeletal features (e.g., the ribcage and spine) pertaining to the subject patient.

Using the ablation simulation platform, the physician is able to view the display 300 and scroll through the views or “slices” of each orientation 304a-d by rolling the wheel of a mouse (user input device) connected to a computer system (e.g., the control system 102 of FIG. 1) running the ablation simulation platform. The physician can scroll through the layers until locating and identifying a disorder or abnormality located in a target tissue region of the subject patient.

FIG. 4 is an enlarged view of the pretreatment scan 302 and, more particularly, an enlarged (close up) view of a section of the axial orientation 304a (FIG. 3). Once uploaded to the ablation simulation platform, as mentioned above, the pretreatment scan 302 may be analyzed by a physician to potentially locate and identify a disorder or abnormality located in a target tissue region 400 present within the subject patient. As provided above, example disorders and abnormalities can include a tumor or an arrhythmia associated with tissue, an organ, a blood vessel, bone, a gland, or any combination thereof. The disorder or abnormality may be remedied by subjecting the target tissue region to an ablation procedure using one or more ablation probes.

Upon identifying the disorder or abnormality and determining that the target tissue region 400 will be subject to an ablation procedure, the user may instruct the ablation simulation platform to enter an ablation planning or simulation mode. Once in the ablation simulation mode, the user may be able to define and graphically identify the target tissue region 400 on the pretreatment scan 302. For example, as illustrated, the user may be able to create a digital target area 402 that encircles and otherwise surrounds or envelops the target tissue region 400. In some embodiments, this may be accomplished by using a mouse cursor superimposed on the pretreatment scan 302 and digitally drawing the digital target area 402 on the pretreatment scan 302. Depending on the input from the user, the digital target area 402 may exhibit any shape or size. In other embodiments, however, the physician may use more complex tools to create the digital target area 402. In yet other embodiments, creation of the digital target area 402 may be automated and based on software instructions programmed into the ablation simulation platform. Example software instructions may include identifying a target tissue region 400 that includes a disorder and creating a digital target area 402 that surrounds (envelops) the target tissue region 400.

Once the digital target area 402 is created (superimposed) on the pretreatment scan 302 and visible on the display 300 (FIG. 3), the user may then proceed to place or plan a pathway (“target path”) for one or more planned ablation probes 404 relative to the target tissue region 400. While only one planned ablation probe 404 is shown in FIG. 4, the planned and simulated placement of more than one planned ablation probe 404 may be undertaken on the ablation simulation platform. As illustrated, the planned ablation probe 404 includes a first or “distal” end 406a and a second or “proximal” end 406b opposite the distal end 406a. Simulating the planned ablation probe 404 effectively sets a target path for the planned ablation probe 404, which may be useful in subsequent placement of an actual ablation probe. Consequently, the distal end 406a may represent the distal end of an actual ablation probe, such as the location of the antenna 208 (FIG. 2) and/or the stylet 218 (FIG. 2), and the proximal end 406b may represent the proximal end of an actual ablation probe, such as the location of the handle 202 (FIG. 2).

As illustrated, the user may set the target path of the planned ablation probe 404 by placing (setting) the distal end 406a of the planned ablation probe 404 at or within the digital target area 402 and fixing the distal end 406a in place. The proximal end 406b may then be selected at a location proximal to the digital target area 402 and outside of the body of the subject patient; e.g., outside of the skin 408 of the subject patient. In other embodiments, however, the user may alternatively place the proximal end 406b first, then the distal end 406a. The ablation simulation platform may be programmed to compute (determine) which end 406a,b is distal and which is proximal based on the orientation of the planned ablation probe 404 to the body (i.e. the distal end 406a is closer to the center of the body. The ablation simulation platform may then generate a line that extends between the distal and proximal ends 406a,b, which represents the target path of the planned ablation probe 404. The line may also represent a simulated or virtual target path that an actual ablation probe may take to reach the digital target area 402.

The user may be able to move either end 406a,b after the planned ablation probe 404 is placed, but may only have the capability to move one end 406a,b at a time. In some applications, the user may adjust the position (orientation) of the proximal end 406b relative to the distal end 406a, which remains stationary. This may occur to obtain an optimized or more efficient target path for the planned ablation probe 404. In some embodiments, as illustrated, the planned ablation probe 404 may be depicted in 2D, but could alternatively be extrapolated in 3D by scrolling through the layers or planes (“slices”) of the pretreatment scan 302.

In some embodiments, the distal and proximal ends 406a,b need not reside in the same layer or plane (“slice”) of the pretreatment scan 302. For example, portions of the planned ablation probe 404 may be broken 410 (e.g., dotted, dashed, etc.) and may represent portions that are out of plane and otherwise not shown in the currently visible layer (“slice”) of the pretreatment scan 302 or otherwise behind (or in front of) the currently visible layer. Moreover, portions of the planned ablation probe 404 may be solid 412 and represent portions that are visible in the currently visible layer of the pretreatment scan 302. In some embodiments, a scan intersection point 414 may be superimposed on the pretreatment scan 302 at a location where the planned ablation probe 404 intersects with the currently visible layer (“slice”). The scan intersection point 414 may be graphically represented in a variety of ways including, but not limited to, a small, solid structure, such as an oval or other geometric shape.

In some embodiments, the ablation simulation platform may be configured to automatically provide a distance measurement 416 between the distal end 406a of the planned ablation probe 404 and a point at which the planned ablation probe 404 exits the skin 408 of the subject patient. The distance measurement 416 may be useful in determining which actual ablation probe may be required to perform the planned ablation procedure. In at least one embodiment, as illustrated, the distance measurement 416 may be depicted as a tick mark perpendicular to the line extending between the distal and proximal ends 406a,b, but could alternatively be depicted in any other perceivable (visible) fashion.

In some embodiments, the ablation simulation platform may be configured and otherwise programmed to identify and display one or more critical pathway regions 418 along the target path of the planned ablation probe 404. The critical pathway region 418 may comprise a location or area in the subject patient that may be impenetrable by an ablation probe and otherwise inadvisable to pass through (penetrate) while attempting to reach the digital target area 402. Said differently, the critical pathway region 418 may comprise critical or high density structures that may be present along the target path of the planned ablation probe 404.

One example of the critical pathway region 418 is a dense or high-density area, such as a section or area of bone (e.g., the rib cage). In such applications, identifying the critical pathway region 418 alerts the user that an actual ablation probe may have to penetrate bone to reach the digital target area 402 if following the planned trajectory. The user, therefore, may desire to alter the target path of the planned ablation probe 404 to avoid the critical pathway region 418. Other examples of the critical pathway region 418 include, but are not limited to, vasculature, a major organ (e.g., the heart, a kidney, etc.), the collecting system in the kidney, portions of the lung, or any combination thereof.

When the target path of the planned ablation probe 404 passes through the critical pathway region 418, the ablation simulation platform may be configured to alert the user. In some embodiments, for example, the critical pathway region 418 may be graphically represented and otherwise superimposed on the pretreatment scan 302 via a visual alert or indication. One example of the visual alert includes changing the color of the portion of the target path that traverses the critical pathway region 418, thereby providing a visual indication of a region or area that may need to be avoided. In some embodiments, the portion of the target path that traverses the critical pathway region 418 may be changed to the color red, for example, but other colors or patters may instead be used, without departing from the scope of the disclosure. If the user decides to reorient the target path away from the critical pathway region 418, the visual alert will be removed (e.g., the color or pattern of the target path will return to a neutral color or pattern).

Another example of the visual alert includes enlarging the portion of the target path that traverses the critical pathway region 418 or superimposing a shape on the target path that traverses the critical pathway region 418. In some embodiments, instead of reorienting the target path of the planned ablation probe 404, the user may be able to place and leave the planned ablation probe 404 intersecting the critical pathway region 418 and proceed with the remaining steps of the planning process.

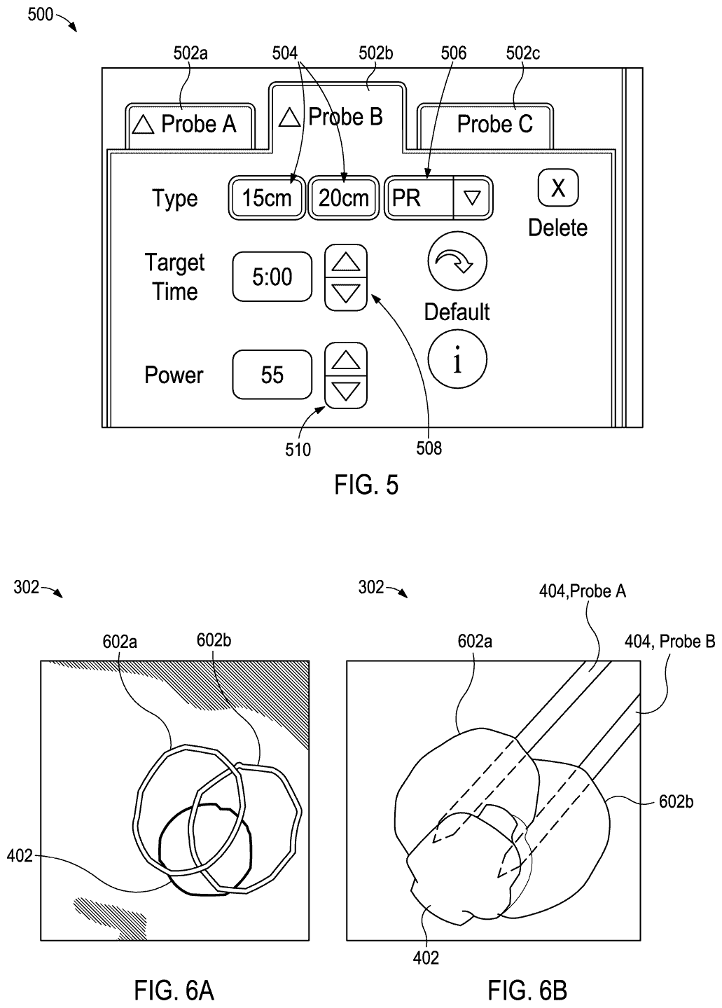

FIG. 5 is an enlarged user interface 500 that may form part of the ablation simulation platform, according to one or more embodiments. In particular, the user interface 500 may form part of or be viewable on the display 300 of FIG. 3, and may allow a user to define and place (plan) one or more planned ablation probes, shown in FIG. 5 as a first planned ablation Probe A, a second planned ablation Probe B, and a third planned ablation Probe C. By clicking on tab 502a for Probe A, the user is able to define parameters (characteristics) for the first planned ablation Probe A, by clicking on tab 502b for Probe B, the user is able to define parameters (characteristics) for the second planned ablation Probe B, and by clicking on tab 502c for Probe C, the user is able to define parameters (characteristics) for the third planned ablation Probe C. The planned ablation Probes A-C may be the same as or similar to the planned ablation probe 404 (FIG. 4), and the user interface 500 may be displayed side-by-side with the pretreatment scan 302 (FIGS. 3 and 4), thereby allowing a user to see in real-time the creation and visual representation of the target path for each planned ablation Probe A-C, based on the selected parameters (characteristics).

The user interface 500 may include various options and buttons that allow a user to select one or more parameters for each planned ablation Probe A-C. Based on the selected parameters, the size and effect of the simulated (predicted) ablation will increase or decrease. For example, the user interface 500 may include one or more buttons 504 that allow a user to select a particular length for each planned ablation Probe A-C. In the illustrated embodiment, for example, a user can select a planned ablation Probe A-C that has a length of 15 cm or 20 cm. In other embodiments, however, the user interface 500 may include additional options allowing a user to select planned ablation Probes A-C that have a length that is less than 15 cm or greater than 20 cm, without departing from the scope of the disclosure. The distance measurement 416 (FIG. 4) superimposed on the pretreatment scan 302 (FIG. 4) may help the user determine what length may be required to reach the digital target area 402 (FIG. 4).

The user interface 500 may also include an option or button 506 that allows the user to select a particular type or design of the planned ablation Probe A-C. The button 506, for example, may comprise a drop down menu button including various selectable probe designs (models) such as, but not limited to, PR=Precision, LK=Liver and Kidney, LN=Lung, and XT=Smaller Gauge Probe. The PR-type probe may comprise an ablation probe designed for more precise ablation treatments, and the LK-type probe may comprise an ablation probe designed for ablation treatments in the liver and/or the kidney. The LN-type probe may comprise an ablation probe designed for operation in the lung, and the XT-type probe may comprise an ablation probe that is thicker and/or stiffer, and thus more robust than the other probe designs (models).

The user interface 500 may also include one or more buttons 508 that allow a user to select or adjust an ablation time; i.e., the time that the ablation probe will be delivering energy (e.g., microwaves). In particular, increasing the ablation time will correspondingly increase the amount of time the planned ablation Probe A-C will be active during the ablation process. Longer ablation times will result in a larger resulting ablated zone.

The user interface 500 may also include one or more buttons 510 that allow a user to adjust the power output (e.g., in watts) of the planned ablation Probes A-C. Higher power output for the planned ablation Probes A-C will result in a larger ablation zone and higher temperatures sooner in the ablation process, and lower power output for the planned ablation Probes A-C will result in a smaller ablation zone and lower temperatures achieved during the ablation.

FIGS. 6A and 6B are enlarged views of the pretreatment scan 302 of FIG. 4 depicting 2D and 3D planned ablation zones, respectively. More specifically, based on the preferences, characteristics, and parameters input into the user interface 500, the user may cause the ablation simulation platform to generate a planned ablation zone corresponding to each planned ablation Probe A-C. In FIG. 6A, the digital target area 402 and first and second planned ablation zones 602a and 602b are superimposed and otherwise overlaid on the pretreatment scan 302 in 2D. In FIG. 6B, the digital target area 402 and the first and second planned ablation zones 602a,b are superimposed and otherwise overlaid on the pretreatment scan 302 in 3D. The planned ablation zones 602a,b represent estimated ablations corresponding to planned ablation Probes A and B, respectively.

The estimated size and position (location) of each planned ablation zone 602a,b is determined by the user-input parameters provided in the user interface 500 (FIG. 5); e.g., the type of probe, the length of the probe, the desired ablation time, and the desired power output (wattage). Moreover, the estimated size of each planned ablation zone 602a,b may also be based on and otherwise dependent on the type of tissue or organ (e.g., liver, lung, kidney, etc.) in which the digital target area 402 is located. More specifically, the size of an ablation zone can depend on the type of tissue or organ in which the ablation probe operates. For example, when using the same ablation probe exhibiting the same parameters, a resulting ablation zone generated in liver may be smaller than a resulting ablation zone generated in lung. Based on ex vivo tissue testing of similar tissues and organs, the ablation simulation platform may be programmed and otherwise configured to generate planned ablation zones with a size (magnitude) that corresponds to the type of tissue in which the ablation occurs and based on the user-input parameters.

Accordingly, the ablation simulation platform may know where the digital target area 402 is located in the subject human, and therefore may know the type of tissue or organ in which the planned ablation zone 602a,b is to be generated. In some applications, for example, the user may indicate the type of tissue and/or the organ they are treating. In other applications, however, the ablation simulation platform may be programmed with an organ segmentation algorithm that automatically segments the tissues/organs and determines in which tissue/organ the probe(s) are in. Based on the type of tissue or organ, and further based on the ex vivo tissue testing, the planned ablation zones 602a,b are generated with a size corresponding to the type of tissue and based on the user provided parameters input via the user interface 500 (FIG. 5).

Referring to FIG. 6B, as mentioned above, the planned ablation Probes A,B may be the same as or similar to the planned ablation probe 404 of FIG. 4. The planned ablation zones 602a,b may be positioned such that the ablation probe 404 passes through the center or major axis of the planned ablation zone 602a,b. The location or position of the planned ablation zone 602a,b along the length of the ablation probe 404 may be determined based on the emission zone of the ablation probe 404.

In the illustrated embodiment, each planned ablation zone 602a,b exhibits a shape that is generally an ellipse or an ellipsoid, but the shapes are not limited to ellipses or ellipsoids. Rather, the planned ablation zones 602a,b can alternatively exhibit any geometry or shape, without departing from the scope of the disclosure. Moreover, in some embodiments, one or both of the planned ablation zones 602a,b may extend across multiple tissue types, organs, or solid structures (e.g., bone). In such embodiments, the geometry and size of the planned ablation zones 602a,b may vary. In such embodiments, the ablation simulation platform may be programmed and otherwise configured to take into account multiple tissue characteristics (e.g., perfusion, conductivity, dielectric, impedance, etc.), organs, or solid structures, and thereby automatically adjust the size and geometry of the planned ablation zones 602a,b.

The planned ablation zones 602a,b allow a user to visualize the spatial relationship between an estimated ablation corresponding to each planned ablation Probe A,B and the digital target area 402. From this comparison, the user can evaluate the potential (predicted) ablation coverage of the digital target area 402 based on the orientation and placement of the Probes A,B. If either (or both) of the planned ablation zones 602a,b does not fully cover (envelop) a portion of the digital target area, plus the desired margin, 402, the user can adjust the orientation and/or the parameters of one or both of the Probes A,B. This can be accomplished by returning to the display 300 (FIG. 3) and the user interface 500 (FIG. 5), where the orientation of the Probes A,B (probe 404 of FIG. 4) can be altered, and/or the probe parameters may be adjusted, such as, but not limited to, probe type, ablation time, and power.

Once one or more of the planned ablation Probes A-C are planned and superimposed on the pretreatment scan 302 (FIGS. 3, 4, 6A-6B), the user (e.g., a physician, a surgeon, a trained technician, etc.) may then proceed to physically place one or more actual ablation probes in the subject patient. Placement of the actual ablation probes may be undertaken using any known or conventional placement techniques, such as using ultrasound or CT, or fluoroscopic image guidance techniques to get actual ablation probes to the desired location (e.g., at or near the digital target area 402).

After the actual ablation probes are placed in the subject patient, a second scan showing the probe position(s) (image) may be obtained of the subject patient using any of the aforementioned imaging technologies; e.g., MRI, CT imaging, PET imaging, ultrasound imaging, nuclear medicine imaging, fluoroscopic imaging, or any combination thereof. The second scan, alternately referred to as an operative or “probe” scan, may detect and display the presence of the actual ablation probes, and the probe scan may then be transferred to the ablation simulation program. In at least one embodiment, the probe scan may be uploaded to the PACS system, and the user may then import the probe scan into the ablation simulation program from the PACS system. Once uploaded to the ablation simulation program, the actual placement of the actual ablation probe(s) may then be automatically detected and overlaid on the pretreatment scan 302 (FIGS. 3, 4, and 6A-6B) to be compared against the digital target area (FIGS. 4 and 6A-6B) and the planned trajectory of the planned ablation Probes A-C(e.g., the planned ablation probe 404 of FIG. 4).

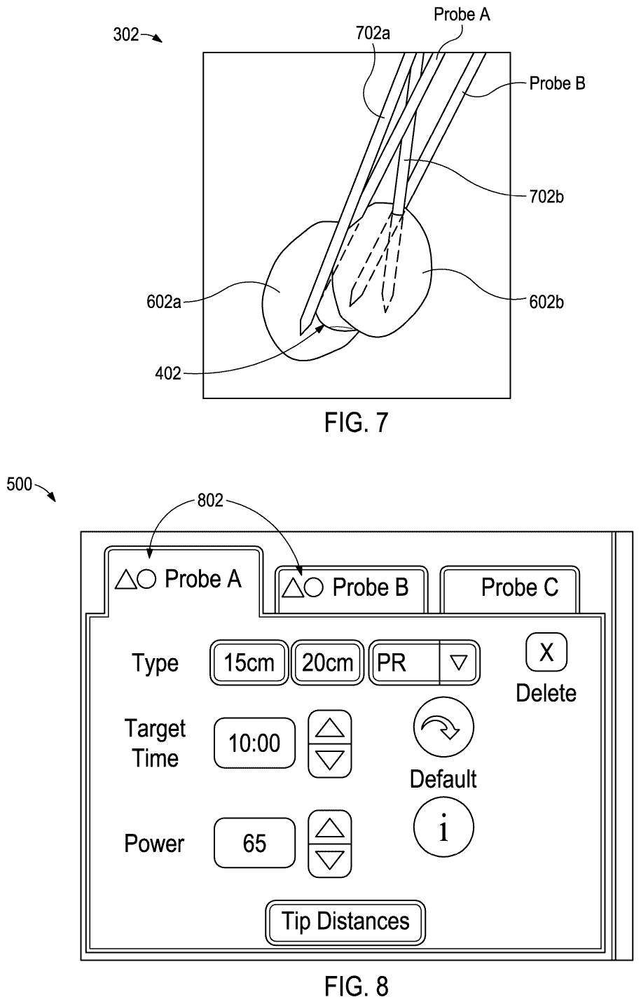

FIG. 7 is an enlarged view of the pretreatment scan 302 overlaid with features detected in a probe scan of the subject patient, according to one or more embodiments. More specifically, the probe scan detected two actual ablation probes placed in the subject patient, shown as a first actual ablation probe 702a and a second actual ablation probe 702b. By importing the probe scan to the ablation simulation program, the first and second actual ablation probes 702a,b may be overlaid on the pretreatment scan 302 and displayed in comparison with the digital target area 402 and the first and second planned ablation Probes A,B. In FIG. 7, the pretreatment scan 302 and the overlaid first and second actual ablation probes 702a,b are shown in 3D, but could otherwise be depicted in 2D, without departing from the scope of the disclosure.

The ablation simulation program may be programmed and otherwise configured to match the actual ablation probes 702a,b with the planned ablation Probes A,B based primarily on proximity to one another. More specifically, based on proximity of the planned ablation Probes A,B to the actual ablation probes 702a,b, the ablation simulation program may be programmed with a matching algorithm that determines (registers) which actual ablation probe 702a,b (if more than one was used) is physically closest to each of the planned ablation Probes A,B. If multiple probes are present, each planned ablation Probe A,B is compared to each detected actual ablation probe 702a,b, and a match is selected based on a smallest value computed by the matching algorithm. If there is only one actual ablation probe and only one planned ablation probe, then the planned and actual ablation probes may be automatically matched.

In some embodiments, the ablation simulation program may apply a weighting based on the distance from the distal end of each probe, since the distal ends of the probes should be closest to the treatment target. Accordingly, the weighting applied in the matching algorithm may be larger when the distal end of the probe is closer to the distal end of the probe being compared 402.

The matching algorithm programmed into the ablation simulation program and used to match the planned ablation Probes A,B to the actual ablation probe 702a,b may also be configured to compare the target path of the planned ablation Probes A,B to a detected path of the actual ablation probes 702a,b. Points are sampled along each planned and actual ablation probe, and the distance to the adjacent probes is computed. The smaller the distance, the better the match. Because the planned ablation Probes A,B are defined in the pretreatment scan 302, and the actual ablation probes 702a,b are in the probe scan (i.e., a different scan), the coordinates of the planned ablation Probes A,B may be moved into the same coordinate system as the actual ablation probes 702a,b. This may be accomplished, for example, by registering one scan to the other and using the registration information to transfer the planned ablation Probes A,B to the probe scan.

Referring briefly to FIG. 8, which depicts an updated version of the user interface 500, once each planned ablation Probe A,B is successfully matched with a corresponding actual ablation probe 702a,b, the user interface 500 may display or include a legend 802 to indicate to the user which planned ablation Probe A,B has been matched to which actual ablation probe 702a,b. In some embodiments, for example, the legend 802 may include color-coded shapes (e.g., triangles, circles, etc.). The specific color of the shapes may correspond to the colors of the planned ablation Probes A,B and the actual ablation probe 702a,b as depicted in the pretreatment scan 302 (FIG. 7). This provides the user with a visual indication of the matched planned and actual ablation probes.

Referring again to FIG. 7, with continued reference to FIG. 8, once the actual ablation probes 702a,b are matched with corresponding planned ablation Probes A,B, the ablation simulation program may then be programmed and otherwise configured to transfer the planned ablation zones 602a,b corresponding to the planned ablation Probes A,B to the matched actual ablation probes 702a,b. Accordingly, the ablation settings and parameters (e.g., ablation time, power, length, etc.) input into the user interface 500 by the user may then be transferred to the actual ablation probe 702a,b, thereby matching the planned ablation zones 602a,b with corresponding actual ablation probe 702a,b.

As will be appreciated, transferring the parameters of the planned ablation zones 602a,b to the actual ablation probes 702a,b allows the user to see planned (estimated) ablation zones when using the actual ablation probes 702a,b, and relative to the digital target area 402. From this comparison, the user can evaluate the potential ablation coverage of the digital target area 402 based on the orientation and placement of the actual ablation probes 702a,b. For example, if either (or both) of the planned ablation zones 602a,b does not fully cover (envelop) a portion of the digital target area plus the desired margin 402, the user can adjust the parameters of one or both of the actual ablation probes 702a,b. This can be accomplished by returning to the user interface 500 and adjusting the probe parameters, such as ablation time and power (wattage). Based on the parameter changes, the size and orientation of the planned ablation zones 602a,b may then be updated in real-time on the display 300 (FIG. 3), thereby allowing the user to verify if the digital target area 402 will be sufficiently ablated during operation. As will be appreciated, this will allow the user to obtain desired ablation results.

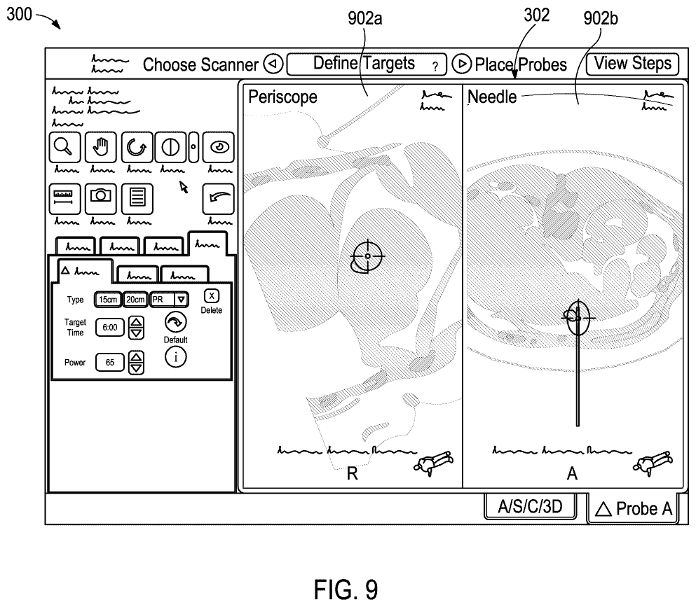

FIG. 9 is another depiction of the display 300 and the pretreatment scan 302, according to one or more additional embodiments. In the illustrated embodiment, the pretreatment scan 302 is depicted in a periscope view 902a and a needle view 902b. The periscope view 902a comprises a series of images which are resampled such that the probe represents the central axis of the images. The needle view 902b comprises a series of images which are resampled such that they are perpendicular to the periscope view images and the probe is normal to the central axis. The periscope and needle views 902a,b may be useful in allowing a physician to look at the anatomy of the subject patient from both views, simultaneously. The periscope and needle views 902a,b help the user view the orientation of the probe to the targeted tissue, and these views may help determine the location of organs and other structures within the body of the subject patient that they want to avoid when placing the actual ablation probes. In some embodiments, the periscope and needle views 902A, B may be accessible and otherwise usable in the ablation simulation program.

Embodiments disclosed herein include:

A. A method of planning a microwave ablation procedure includes the steps of uploading a pretreatment scan to a control system of an energy delivery system, displaying the pretreatment scan on a display of the control system, analyzing the pretreatment scan and identifying a target tissue region that includes a disorder, creating and graphically displaying a digital target area on the pretreatment scan and relative to the target tissue region, planning and graphically depicting on the pretreatment scan a target path for one or more planned ablation probes relative to the target tissue region, creating a planned ablation zone for each planned ablation probe based on one or more user-selected parameters, placing one or more actual ablation probes in the subject patient relative to the target tissue region, obtaining a probe scan of the subject patient that detects the one or more actual ablation probes, and assigning the planned ablation zone for the one or more planned ablation probes to the one or more actual probes.

B. A non-transitory medium readable by a processor and storing instructions for execution by the processor for performing a method including the steps of uploading a pretreatment scan to a control system of an energy delivery system, displaying the pretreatment scan on a display of the control system, creating and graphically displaying a digital target area on the pretreatment scan and relative to the target tissue region, the target tissue region including a disorder identified in the pretreatment scan, planning and graphically depicting on the pretreatment scan a target path for one or more planned ablation probes relative to the target tissue region, creating a planned ablation zone for each planned ablation probe based on one or more user-selected parameters, obtaining a probe scan of the subject patient after placement of one or more actual ablation probes in the subject patient relative to the target tissue region, the probe scan detecting the one or more actual ablation probes, and assigning the planned ablation zone for the one or more planned ablation probes to the one or more actual probes.

Each of embodiments A and B may have one or more of the following additional elements in any combination: Element 1: wherein the disorder comprises at least one of a tumor or arrhythmia associated with tissue, an organ, a gland, a blood vessel, bone, and any combination thereof. Element 2: wherein creating the digital target area comprises digitally drawing the digital target area on the pretreatment scan. Element 3: wherein planning the target path for the one or more planned ablation probes comprises setting a distal end of the one or more planned ablation probes at or within the digital target area, setting a proximal end of the one or more planned ablation probes at a location proximal to the digital target area and outside of a body of the subject patient, and generating and graphically depicting a line extending between the distal and proximal ends. Element 4: further comprising graphically depicting broken portions of the line corresponding to portions of the target path that reside behind, or in front of, a plane of the pretreatment scan, and graphically depicting solid portions of the line corresponding to portions of the target path that reside in the plane of the pretreatment scan. Element 5: further comprising identifying and displaying one or more critical pathway regions along the target path for at least one of the one or more planned ablation probes. Element 6: wherein the one or more critical pathway regions are selected from the group consisting of a dense or high-density area, vasculature, a major organ, a collecting system in the kidney, portions of the lung, and any combination thereof. Element 7: wherein the one or more user-selected parameters are selected from the group consisting of a length of the one or more planned ablation probes, a model of the one or more planned ablation probes, a desired ablation time, and a desired power output for the one or more planned ablation probes. Element 8: further comprising adjusting the one or more user-selected parameters of at least one of the one or more planned ablation probes and thereby altering a size of the planned ablation zone. Element 9: further comprising creating the planned ablation zone for each planned ablation based on a type of tissue or organ in which the digital target area is located. Element 10: wherein obtaining the probe scan of the subject patient comprises uploading the probe scan to the control system, overlaying placement of the one or more actual ablation probes on the pretreatment scan, and comparing placement of the one or more actual ablation probes against the digital target area and the target path of the one or more planned ablation probes. Element 11: wherein assigning the planned ablation zone for the one or more planned ablation probes to the one or more actual probes comprises matching the one or more actual ablation probes to the one or more planned ablation probes based on a matching algorithm, and transferring the one or more user-selected parameters of the planned ablation zones to the one or more actual ablation probes. Element 12: further comprising adjusting the one or more user-selected parameters and thereby altering a size of the planned ablation zone relative to the digital target area.

Element 13: wherein planning the target path for the one or more planned ablation probes comprises setting a distal end of the one or more planned ablation probes at or within the digital target area, setting a proximal end of the one or more planned ablation probes at a location proximal to the digital target area and outside of a body of the subject patient, and generating and graphically depicting a line extending between the distal and proximal ends. Element 14: further comprising graphically depicting broken portions of the line corresponding to portions of the target path that reside behind, or in front of, a plane of the pretreatment scan, and graphically depicting solid portions of the line corresponding to portions of the target path that reside in the plane of the pretreatment scan. Element 15: further comprising calculating a distance from the distal end to a point at which the target path exits a skin of the subject patient, and displaying a numerical value of the distance on the pretreatment scan. Element 16: further comprising identifying and displaying one or more critical pathway regions along the target path for at least one of the one or more planned ablation probes. Element 17: further comprising creating the planned ablation zone for each planned ablation based on a type of tissue or organ in which the digital target area is located. Element 18: wherein obtaining the probe scan of the subject patient comprises uploading the probe scan to the control system, overlaying placement of the one or more actual ablation probes on the pretreatment scan, and comparing placement of the one or more actual ablation probes against the digital target area and the target path of the one or more planned ablation probes. Element 19: wherein assigning the planned ablation zone for the one or more planned ablation probes to the one or more actual probes comprises matching the one or more actual ablation probes to the one or more planned ablation probes based on proximity, and transferring the one or more user-selected parameters of the planned ablation zones to the one or more actual ablation probes.

By way of non-limiting example, exemplary combinations applicable to A and B include: Element 3 with Element 4; Element 5 with Element 6; Element 7 with Element 8; Element 11 with Element 12; Element 13 with Element 14; and Element 14 with Element 15.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, uphole, downhole and the like are used in relation to the illustrative embodiments as they are depicted in the figures, the upward direction being toward the top of the corresponding figure and the downward direction being toward the bottom of the corresponding figure.

What is claimed is:

1. A method of planning a microwave ablation procedure, comprising:
uploading a pretreatment scan to a control system of an energy delivery system;
displaying the pretreatment scan on a display of the control system;
analyzing the pretreatment scan and identifying a target tissue region that includes a disorder;
creating and graphically displaying a digital target area on the pretreatment scan and relative to the target tissue region;
planning and graphically depicting on the pretreatment scan a target path for one or more planned ablation probes relative to the target tissue region;
creating a planned ablation zone for each planned ablation probe based on one or more user-selected parameters;
placing one or more actual ablation probes in the subject patient relative to the target tissue region;
obtaining a probe scan of the subject patient that detects the one or more actual ablation probes; and
assigning the planned ablation zone for the one or more planned ablation probes to the one or more actual probes.

2. The method of claim 1, wherein the disorder comprises at least one of a tumor or an arrhythmia associated with tissue, an organ, a gland, a blood vessel, bone, and any combination thereof.

3. The method of claim 1, wherein creating the digital target area comprises digitally drawing the digital target area on the pretreatment scan.

4. The method of claim 1, wherein planning the target path for the one or more planned ablation probes comprises:
setting a distal end of the one or more planned ablation probes at or within the digital target area;
setting a proximal end of the one or more planned ablation probes at a location proximal to the digital target area and outside of a body of the subject patient; and
generating and graphically depicting a line extending between the distal and proximal ends.

5. The method of claim 4, further comprising:
graphically depicting broken portions of the line corresponding to portions of the target path that reside behind, or in front of, a plane of the pretreatment scan; and
graphically depicting solid portions of the line corresponding to portions of the target path that reside in the plane of the pretreatment scan.

6. The method of claim 1, further comprising identifying and displaying one or more critical pathway regions along the target path for at least one of the one or more planned ablation probes.

7. The method of claim 6, wherein the one or more critical pathway regions are selected from the group consisting of a dense or high-density area, vasculature, a major organ, a collecting system in the kidney, portions of the lung, and any combination thereof.

8. The method of claim 1, wherein the one or more user-selected parameters are selected from the group consisting of a length of the one or more planned ablation probes, a model of the one or more planned ablation probes, a desired ablation time, and a desired power output for the one or more planned ablation probes.

9. The method of claim 8, further comprising adjusting the one or more user-selected parameters of at least one of the one or more planned ablation probes and thereby altering a size of the planned ablation zone.

10. The method of claim 1, further comprising creating the planned ablation zone for each planned ablation based on a type of tissue or organ in which the digital target area is located.

11. The method of claim 1, wherein obtaining the probe scan of the subject patient comprises:
uploading the probe scan to the control system;
overlaying placement of the one or more actual ablation probes on the pretreatment scan; and
comparing placement of the one or more actual ablation probes against the digital target area and the target path of the one or more planned ablation probes.

12. The method of claim 1, wherein assigning the planned ablation zone for the one or more planned ablation probes to the one or more actual probes comprises:
matching the one or more actual ablation probes to the one or more planned ablation probes based on a matching algorithm; and
transferring the one or more user-selected parameters of the planned ablation zones to the one or more actual ablation probes.

13. The method of claim 12, further comprising adjusting the one or more user-selected parameters and thereby altering a size of the planned ablation zone relative to the digital target area.

14. A non-transitory medium readable by a processor and storing instructions for execution by the processor for performing a method comprising:
uploading a pretreatment scan to a control system of an energy delivery system;
displaying the pretreatment scan on a display of the control system;
creating and graphically displaying a digital target area on the pretreatment scan and relative to the target tissue region, the target tissue region including a disorder identified in the pretreatment scan;
planning and graphically depicting on the pretreatment scan a target path for one or more planned ablation probes relative to the target tissue region;
creating a planned ablation zone for each planned ablation probe based on one or more user-selected parameters;
obtaining a probe scan of the subject patient after placement of one or more actual ablation probes in the subject patient relative to the target tissue region, the probe scan detecting the one or more actual ablation probes; and
assigning the planned ablation zone for the one or more planned ablation probes to the one or more actual probes.

15. The non-transitory medium of claim 14, wherein planning the target path for the one or more planned ablation probes comprises:
setting a distal end of the one or more planned ablation probes at or within the digital target area;
setting a proximal end of the one or more planned ablation probes at a location proximal to the digital target area and outside of a body of the subject patient; and
generating and graphically depicting a line extending between the distal and proximal ends.

16. The non-transitory medium of claim 15, further comprising:
graphically depicting broken portions of the line corresponding to portions of the target path that reside behind, or in front of, a plane of the pretreatment scan; and
graphically depicting solid portions of the line corresponding to portions of the target path that reside in the plane of the pretreatment scan.

17. The non-transitory medium of claim 16, further comprising:
calculating a distance from the distal end to a point at which the target path exits a skin of the subject patient; and
displaying a numerical value of the distance on the pretreatment scan.

18. The non-transitory medium of claim 14, further comprising identifying and displaying one or more critical pathway regions along the target path for at least one of the one or more planned ablation probes.

19. The non-transitory medium of claim 14, further comprising creating the planned ablation zone for each planned ablation based on a type of tissue or organ in which the digital target area is located.

20. The non-transitory medium of claim 14, wherein obtaining the probe scan of the subject patient comprises:
uploading the probe scan to the control system;
overlaying placement of the one or more actual ablation probes on the pretreatment scan; and
comparing placement of the one or more actual ablation probes against the digital target area and the target path of the one or more planned ablation probes.

21. The non-transitory medium of claim 14, wherein assigning the planned ablation zone for the one or more planned ablation probes to the one or more actual probes comprises:
matching the one or more actual ablation probes to the one or more planned ablation probes based on proximity; and
transferring the one or more user-selected parameters of the planned ablation zones to the one or more actual ablation probes.
